(19) United States
(12) Patent Application Publication
(10) Pub. No.: US 2005/0084863 A1
(43) Pub. Date: Apr. 21, 2005

(54) Removal of nucleic acid contaminants

(75) Inventor: Suzanne Margaret Price, Kardinya (AU)

Correspondence Address: DAVIDSON, DAVIDSON & KAPPEL, LLC, 485 SEVENTH AVENUE, 14TH FLOOR, NEW YORK, NY, 10018, US

(73) Assignee: Biomolecular Research Solutions Pty Ltd, Kardinya (AU)

(21) Appl. No.: 10/505,213

(22) PCT Filed: Feb. 20, 2003
(86) PCT No.: PCT/AU03/00213

(30) Foreign Application Priority Data
Feb. 20, 2002 (AU) PS 0642

(51) Int. Cl. C12Q 1/68; C12P 19/34

(52) U.S. Cl. 435/6; 435/91.2

(57) ABSTRACT

A method of analysing a nucleic acid sample obtained from a site comprising the step of pretreating the sample to remove or inactivate contaminating nucleic acids originating from the site. The pre-treatment may comprise chemical, enzyme or a physical treatment that acts to selectively or preferentially remove or inactivate the contaminating nucleic acids.

### FIELD OF THE INVENTION

The present invention relates to improved methods for collecting and analysing nucleic acid samples such as nucleic acid samples of forensic value from crime scenes. The present invention also relates to databases containing data obtained using the improved methods.

### BACKGROUND OF THE INVENTION

The analysis of DNA and in particular DNA identification such as DNA profiling, DNA fingerprinting and genetic profiling (hereinafter referred to as “DNA profiling”), is used in many areas of research and commercial activity including agriculture, veterinary science, medicine and forensics. In agriculture and veterinary science, DNA profiling is used to identify plant and animal genotypes for breeding and identification purposes and in medical science DNA profiling is used for various purposes including identification of related individuals.

DNA profiling has also become an important tool in forensic science and law enforcement. In DNA forensics, DNA isolated from crime scenes can be amplified and visualised using techniques such as polymerase chain reaction (“PCR”) and gel electrophoresis and the resulting profile or “fingerprint” can be used to place a suspect at a crime scene.

The methods used to analyse DNA, such as PCR amplification, are simple, quick and highly sensitive so they can be carried out using small samples or samples that have been partially degraded. However, the sensitivity of DNA analysis techniques also has potential disadvantages. For example, strict contamination control is essential when undertaking analysis using PCR as contaminants in the starting sample will also be amplified. This is particularly so when the contaminants are amplicons derived from PCR as these are amplified with high efficiency during PCR. Consequently, forensic and other testing laboratories go to considerable effort to prevent the contamination of samples during both the collection and processing stages.

When samples are contaminated and the source of the contamination can be readily identified (e.g. the person collecting the sample or running the PCR) samples can be taken from those individuals and the PCR bands generated by that individual subtracted from the test results to correct for the contamination. In a crime scene where multiple individuals may be present, most contaminating individuals (samples not belonging to the perpetrator/s) can be identified and the effects of that contamination eliminated from further analysis.

However, to date it has not been recognised that DNA samples could be purposefully contaminated with the intention of confounding future DNA analysis. For example, microsatellite PCR amplicons generated by the use of a commercially available kit on a standard DNA control or random tissue sample (hereafter referred to as “perfect amplicons”) could be added to water or another solvent and used to contaminate a sample or the area from which a sample for future DNA analysis is to be taken. In the example of a contaminated crime scene, these perfect amplicons would be collected along with the forensic sample during the collection of samples for forensic analysis from the scene. These perfect amplicons would be efficiently isolated using current DNA extraction methods widely used in DNA forensics and may be present in a vast excess over the DNA of true forensic value from the crime scene. Furthermore, being short perfect amplicons, they would be amplified with greater efficiency than any genomic DNA present. Upon amplification the resulting profile would consist almost entirely of the contaminating perfect amplicon DNA. Depending on the nature of the contaminating amplicons and their concentration in the sample(s), the resultant profile may be indistinguishable from a real profile, or may render the identification of the genuine profile of the forensic sample difficult or impossible to determine.

Although current forensic testing usually uses PCR amplification of selected microsatellite regions from the forensic sample nucleic acid, other methods in use or development such as mitochondrial DNA sequencing, single nucleotide polymorphism analysis, low copy number PCR and other methods known to those familiar with DNA analysis methods are also susceptible to this form of contamination.

When not accounted for in the testing process, the potential for contamination of this nature compromises the validity of the DNA analysis and substantially limits the strength of any conclusions drawn therefrom. For example, crime scenes could be contaminated with nucleic acid with a view to confounding future forensic analysis and limiting the legal value of the DNA analysis results used in court proceedings.

At present, the techniques used to analyse nucleic acid samples for forensic purposes do not reliably distinguish between nucleic acids added to contaminate the sample and the true target nucleic acids in a sample. Laboratory procedures currently used are designed to minimise contamination of samples in the laboratory and will not be effective in removing contamination when contaminated samples are presented to the laboratory for analysis.

The most common and widely used system to reduce or remove contamination with PCR derived amplicons relies on the incorporation of DNA nucleotide analogues such as deoxy-uracil triphosphate (dUTP) into DNA during PCR amplification. However, this method is also designed to prevent laboratory cross contamination and does not address the problems encountered when a sample has been contaminated with nucleic acid containing deoxy-thymine triphosphate (thymine, dTTP) instead of dUTP. Consequently this method of contamination control is easily avoided and is not effective in removing contamination when dTTP containing samples are presented to the laboratory for analysis.

Thus, the present invention seeks to provide methods that deal with the previously unrecognised problem of reliably detecting the presence of contamination and processing nucleic acid samples that have the potential of being, or have been, purposefully contaminated to remove the contaminant.

### SUMMARY OF THE INVENTION

The present invention provides a method of analysing a nucleic acid sample obtained from a site comprising the step of pretreating the sample to remove or inactivate contaminating nucleic acids originating from the site.

The present invention also provides a method of screening a nucleic acid sample for contaminants that have been purposefully introduced into the sample, the method comprising the step of treating the sample to detect the contaminants.

The methods of the present invention may be broadly applied and in particular may be applied to forensics and animal, plant and human nucleic acid testing.

### BRIEF DESCRIPTION OF THE DRAWINGS

FIG. 1 depicts a gel electrophoresis of various PCR amplifications of uncontaminated and contaminated samples;

FIG. 2 depicts a gel electrophoresis of another series of PCR amplifications of treated and untreated contaminated samples;

FIG. 3 depicts a gel electrophoresis of various PCR amplifications of uncontaminated and contaminated samples

FIG. 4 depicts a gel electrophoresis of various PCR amplifications of uncontaminated and contaminated samples;

FIG. 5 depicts a gel electrophoresis of various PCR amplifications of uncontaminated and contaminated samples;

FIG. 6 depicts a gel electrophoresis of various PCR amplifications of contaminated samples that have been treated according to one embodiment of the invention; and

FIG. 7 depicts a gel electrophoresis of various PCR amplifications of contaminated samples that have been treated according to one embodiment of the invention.

### DETAILED DESCRIPTION OF THE INVENTION

The present invention provides a method of analysing a nucleic acid sample obtained from a site comprising the step of pretreating the sample to remove or inactivate contaminating nucleic acids originating from the site.

For the purposes of the present invention the phrase “contaminating nucleic acid/s” is defined as nucleic acid that has been introduced to a site or a sample to confound future analysis of target nucleic acids present at the site or in the sample. The contaminating nucleic acid may be cell bound, free or substantially free from other cell components and may be deoxyribonucleic acid (DNA), ribonucleic acid (RNA), protein nucleic acid (PNA), locked nucleic acid (LNA) or any other nucleic acid containing composition such as those containing natural nucleotides (e.g. dATP, dCTP, dTTP, dUTP, dGTP) or nucleotide/nucleoside analogues that are capable of detection during testing procedures.

When the contaminating nucleic acid is free or substantially free from other cell components it may be in a form that is particularly well adapted for amplification via PCR or some other amplification process that is used in forensic analysis. One particular example of this type of contaminating nucleic acid is an amplicon derived from a PCR or another DNA amplification process and in particular a degradation resistant amplicon that has been specifically designed to persist at a site or in a sample. Synthetic DNA, RNA or PNA may also be used.

The contamination addressed by the present invention may confound any nucleic acid analysis protocol where samples may be contaminated. Thus, while specific mention is made herein of PCR, it will be appreciated that the same contamination could be used to alter the results of other analysis methods such as, but not limited to, mitochondrial DNA sequencing, single nucleotide polymorphism (SNP) analysis and low copy number PCR.

When the contaminating nucleic acid is cell bound it may also be in a form that is particularly well adapted for amplification via PCR or some other amplification process that is used for analysing nucleic acids. One particular example of this type of contaminating nucleic acid is a bacterial preparation where the bacteria have been engineered to contain one or more multicopy plasmids each comprising one or more amplicons able to be amplified during standard forensic PCR processes.

The pre-treatment may be varied depending on the nature of the contaminating nucleic acids that require removal or inactivation. Thus, when the contaminating nucleic acids are free or substantially free from other cell components, the pre-treatment may comprise treating the sample to preferentially remove or inactivate nucleic acids that are free or substantially free from other cell components. Such treatments may be one or more treatments selected from the group comprising: (i) enzymic treatments such as contacting the sample with enzymes that preferentially breakdown free nucleic acids e.g. DNAses, RNAses, exonucleases and endonucleases; (ii) physical treatments that remove free contaminating nucleic acid from the sample based on differences between physical characteristics of the contaminating nucleic acid and the target nucleic acid such as charge, density, weight and size and the actual techniques used may be selected from the group comprising centrifugation (e.g. with centricon 100 columns), washing, filtration and chromatography such a gel filtration chromatography; or (iii) chemical treatments such as the use of sodium hydroxide, sodium hypochlorite, sodium metabisulfite, sodium bisulfite or ammonium metabisulphite, detergents (e.g. Tween 20, Alcanox or SDS) as well as proprietary products designed to remove nucleic acids form surfaces such as DNA Zap, RNA Zap, DNA Free or RNA Free (Ambion Inc., Austin, Tex., USA).

When the contaminating nucleic acids are free from other cell components then the pre-treatment may comprise contacting the sample with nucleic acid probes that preferentially bind to the contaminating nucleic acids and render them removable from the sample. This is particularly appropriate for the removal of contaminating nucleic acids in the form of PCR derived amplicons.

Thus, the present invention also provides a method of analysing a nucleic acid sample obtained from a site comprising the step of contacting the sample with a nucleic acid probe that preferentially binds to the contaminating nucleic acids and renders them removable from the sample.

The choice of probe is entirely dependent on the nature of the contaminant. However, it is envisaged that the most common contaminants will be derived from the commercially available forensic DNA test kits and in particular the positive controls that can be readily amplified via PCR. Thus, the probes may be designed to specifically hybridise to the amplification product of the positive control from a proprietary kit. In the event that a new contaminant is produced then it would be necessary to first characterise the contaminant to enable appropriate probes to be designed for use in the method.

The nucleic acid probe may be labelled to aid in its removal from the sample. Suitable labels include biotin/streptavidin.

In one particular form of the invention the contaminating nucleic acid bound to the labelled probe is removed through the use of a chromatography column adapted to specifically bind the label.

When the contaminating nucleic acids are cell bound or otherwise cell associated in a way that prevents or hampers their removal or inactivation, such as if contained in bacterial cells, then additional pre-treatments may be required. For example, when the contaminating nucleic acids are contained within bacterial cells, an additional step to selectively lyse the bacterial cells may be employed. Once the bacterial cells have been lysed the techniques discussed above could be used to complete the pre-treatment. Contaminants in the form of bacterial cells may also be removed by using a filter that selectively removes the bacterial cells from the sample.

Once the sample has been pre-treated it can be treated according to standard techniques for nucleic acid analysis. Thus, the present invention also provides a method of analysing a nucleic acid sample obtained from a site comprising the steps of:
(i) pre-treating the sample to remove or inactivate contaminating nucleic acids originating from the site; and
(ii) characterising the target nucleic acids in the sample.

The nucleic acids in the sample can be characterised by any one of a range of techniques that are presently in use in the field. These techniques generally involve isolating the target nucleic acid and then treating it such that it can be conveniently characterised. These techniques and procedures are well known by those skilled in the art.

The target nucleic acid may be isolated using standard extraction protocols that involve lysing the cells to free the nucleic acid and then separating the nucleic acid from other cellular material. Once isolated, to increase the amount of target nucleic acid, the target nucleic acid may be selectively amplified using PCR or some other technique that is able to replicate the target DNA to increase the amount available for further analysis. Once amplified the target nucleic acid can be visualised using gel electrophoresis. Proprietary DNA profiling or fingerprinting kits can also be used to perform this part of the method.

Screening Methods

Rather than applying the method of the present invention to all samples taken for nucleic acid analysis, it may be preferred to screen samples for contamination prior to nucleic acid analysis. By applying this method, samples that have been contaminated can be identified and handled accordingly.

Thus, the present invention also provides a method of screening a nucleic acid sample for contaminants that have been purposefully introduced into the sample, the method comprising the step of treating the sample to locate the contaminants.

Various treatments may be applied to a sample to screen for contaminants including the use of a detectable probe designed to selectively hybridise to the contaminant. As indicated above, it is expected the most common contaminants will be sourced from commercial DNA analysis kits so the design of probes for this purpose will be routine to those skilled in the art. Alternatively, the wash solutions, filtrate, chromatography column eluate or other products resulting from the procedures used to remove potential contaminants could be tested for the presence of the contaminants.

Databases

The method of the present invention allows for the accurate identification of nucleic acids and counters the effects of contaminants that may have been introduced into a sample with a view to confounding their analysis. DNA fingerprint databases currently in existence include fingerprints that have been determined using methods that do not account for the potential problems of contamination. Given the possibility of contamination, the conclusions drawn from fingerprints in the current databases may be queried. This could be a particular problem in court proceedings where DNA fingerprint evidence has been used to identify a perpetrator. It is possible that DNA analysis performed with protocols that do not account for purposeful contamination may be held inadmissible.

Thus, the present invention also provides a database comprising the results of at least one analysis generated from a method according to the present invention, such as DNA fingerprint.

Preferably, the database is computerised for ease of use and comprises fingerprints of known perpetrators. However, the database can contain any data obtained through the use of the method of the present invention.

Kits

The method of the present invention may be conveniently performed using a kit comprising a series of reagents necessary to carry out the method. Thus, the present invention also provides a nucleic acid analysis kit comprising a means to remove a nucleic acid contaminant from a sample to be subjected to analysis.

The means may be varied and includes those discussed herein such as labelled probe adapted to bind to the contaminant and thus aid in its removal. Alternatively, the means may comprise an enzyme or chemical that can be added to the sample and inactivate of remove the contaminant preferentially or selectively relative to the target nucleic acid.

The method of the present invention is generally applicable to methods for identifying or analysing nucleic acid samples. Described hereunder, are particular applications that demonstrate the broad application of the present invention.

Forensics

The method of the present invention may be of particular use in the analysis of target nucleic acid obtained from crime scenes. Thus, the present invention also provides a method of analysing a nucleic acid sample obtained from a site in the form of a crime scene comprising the step of pretreating the sample to remove or inactivate contaminating nucleic acids originating from the crime scene.

For the purposes of the present invention the phrase “crime scene” is defined to include sites where a crime has been committed or other sites away from the crime scene, where nucleic acid of forensic value relevant to the crime may be found.

In addition to carrying out the method of analysis described above in relation to samples taken from a crime scene it may also be useful when assessing crime scenes to screen the crime scene for contaminating nucleic acid. To date, as this potential problem has not been recognised no specific screening takes place.

Thus, the present invention also provides a method of analysing a crime scene comprising the step of screening the crime scene for contaminating nucleic acids.

The screening step may be carried out by any means apparent to those skilled in the art for detecting contaminating nucleic acids at a crime scene. One such technique involves taking a sample from a point in the crime scene that would not normally contain nucleic acids. This form of screen is particularly appropriate for locating contaminating nucleic acids, such as amplicons, that have been sprayed in liquid form at a crime scene.

In such situations contamination of crime scenes by perfect amplicon material can be detected by testing samples collected from areas within the crime scene surrounding areas in which target nucleic acids are located e.g. sections of walls or floors adjacent a blood stain. The generation of significant DNA profiles from such areas that would not be expected to contain high levels of DNA may indicate a contaminated crime scene.

In situations where individual tissue samples (eg blood, hair etc) have been contaminated by perfect amplicons, contamination may be detected by hybridisation with microsatellite probes or amplification with primers that bind to the primer sequences of known fingerprinting kits, under conditions such that the cells in the tissue sample are not disrupted sufficiently to release significant amounts of genomic DNA (e.g. reduced temperatures).

Veterinary

The method of the present invention may also be applied to the analysis of target nucleic acid obtained from animals. Thus, the present invention also provides a method of analysing a nucleic acid sample obtained from a site in the form of an animal comprising the step of pretreating the sample to remove or inactivate contaminating nucleic acids.

Nucleic acid samples from animals are analysed for a wide range of purposes. Animals of a particular species or breed may be assessed by a potential buyer or breeder to confirm their genotype. Furthermore, commercial herds or products therefrom such as meat may require analysis to assess if they qualify for a government sponsored subsidy or that they meet certain regulatory requirements, such as GMO's or quarantine standards. In all of these situations there is a motive for a person to tamper with the samples for monetary or some other gain.

Agriculture

The method of the present invention may also be applied to the analysis of target nucleic acid obtained from plants. Thus, the present invention also provides a method of analysing a nucleic acid sample obtained from a site in the form of a plant such as a seed comprising the step of pretreating the sample to remove or inactivate contaminating nucleic acids.

Nucleic acid samples from plants are analysed for a wide range of purposes. Plants of a particular species or variety may be assessed by a potential buyer or breeder to confirm their genotype. Furthermore, commercial crops or products therefrom may require analysis to assess if they qualify for a government sponsored subsidy or that they meet certain regulatory requirements, such as GMO's or quarantine standards. In all of these situations there is a motive for a person to tamper with the samples for monetary or some other gain.

Parentage Testing

The method of the present invention may also be applied to the analysis of target nucleic acid obtained from humans for assessing parentage. Thus, the present invention also provides a method of analysing a nucleic acid sample obtained from a site in the form of a human comprising the step of pretreating the sample to remove or inactivate contaminating nucleic acids.

There are obvious motives for a person to tamper with a sample taken for assessment of parentage. Whilst the mechanics of tampering with samples for this purpose may be slightly more complicated, the method of the present invention also solves the problems that could lead to the abuse of this form of nucleic acid testing.

The invention will now be described with reference to two examples. The description of the examples is in no way limiting on the more general description of the invention in the preceding paragraphs.

### EXAMPLES

### Example 1

### Demonstration of PCR Amplification of Contaminating Microsatellite PCR Products During Amplification of Genomic DNA Microsatellite Loci

Materials and Methods:

Template genomic DNA was isolated from muscle tissue of feral cat GD450. This cat carries alleles 25/21 at the locus FCA 69H (GenBank AF130500) carried by the cat chromosome B4. Genomic DNA was extracted from 4.5 mg of muscle tissue using the MasterPure™ DNA Purification Kit (Epicentre Technologies, Madison, Wis., US). The manufacturers recommended protocol for tissue extraction was followed with the exceptions that digestion of the tissue sample with Proteinase K was carried out overnight at 65° C. and protein was pelleted by centrifugation at 4° C.

Three different contaminants were produced by PCR amplification of selected microsatellite loci from genomic DNA isolated from tissue of 3 feral cats:
Cat MV1 (alleles 25/11): allele 25 in common with GD450
Cat MV4 (alleles 31/21): allele 21 in common with GD450
Cat MV5 (alleles 27/11): no allele in common with GD450

The contaminant microsatellite PCR products were approximated to have a final concentration of 50 ng/μl.

PCR conditions and primer sequences used in this study are given in Menotti-Raymond, M.; David, V. A.; Lyons, L. A.; Schaffer, A. A.; Tomlin, J. F.; Hutton, M. K.; O'Brien, S. J. (1999). A Genetic Linkage Map of Microsatellites in the Domestic Cat (Felis catus). Genomics 57 (1), 9-23. Medline 99208656.

Reactions containing 1 μl of GD450 genomic DNA and various dilutions of single or mixed contaminant microsatellite PCR products were prepared as described in Table 1.

TABLE 1

| Gel | Genomic | | Contaminant dilution | |
|---|---|---|---|---|
| lane | DNA Vol. | Contaminant | (template Vol.) | Comments |
| 1 | 1 ul | None | n/a | GD450 Positive control |
| 2 | 1 ul | MV1 | 10⁻¹⁵ (1 ul) | GD450 only |
| 3 | 1 ul | MV1 | 10⁻¹² (1 ul) | GD450 only |
| 4 | 1 ul | MV1 | 10⁻⁹ (1 ul) | GD450 only |
| 5 | 1 ul | MV1 | 10⁻⁶ (1 ul) | GD450/MV1 mixed profile |
| 6 | 1 ul | MV1 | 10⁻³ (1 ul) | GD450/MV1 mixed profile |
| 7 | 1 ul | MV1 | 1 (1 ul) | GD450/MV1 mixed profile |
| 8 | 1 ul | MV4 | 10⁻¹⁵ (1 ul) | GD 450 only |
| 9 | 1 ul | MV4 | 10⁻¹² (1 ul) | GD450 only |
| 10 | 1 ul | MV4 | 10⁻⁹ (1 ul) | GD 450 only |
| 11 | 1 ul | MV4 | 10⁻⁶ (1 ul) | GD450 only |
| 12 | 1 ul | MV4 | 10⁻³ (1 ul) | GD450/MV4 mixed profile |
| 13 | 1 ul | MV4 | 1 (1 ul) | GD450/MV4 mixed profile |
| 14 | 1 ul | MV5 | 10⁻¹⁵ (1 ul) | GD450 only |
| 15 | 1 ul | MV5 | 10⁻¹² (1 ul) | GD450 only |
| 16 | 1 ul | MV5 | 10⁻⁹ (1 ul) | GD450 only |
| 17 | 1 ul | MV5 | 10⁻⁶ (1 ul) | GD450/MV5 mixed profile |
| 18 | 1 ul | MV5 | 10⁻³ (1 ul) | GD450/MV5 mixed profile |
| 19 | 1 ul | MV5 | 1 (1 ul) | GD450/MV5 mixed profile |
| 20 | 1 ul | Lanes 20-25 | 10⁻¹⁵ (1 ul each) | Amplification failure |
| 21 | 1 ul | Mix of: | 10⁻¹² (1 ul each) | Amplification failure |
| 22 | 1 ul | MV1 + MV4 + MV5 | 10⁻⁹ (1 ul each) | Amplification failure |
| 23 | 1 ul | | 10⁻⁶ (1 ul each) | Amplification failure |
| 24 | 1 ul | | 10⁻³ (1 ul each) | GD450/MV1/MV4/MV5 |
| | | | | profile |
| 25 | 1 ul | | 1 (1 ul each) | Too much template |
| 26 | none | none | n/a | No DNA Negative control |

Results

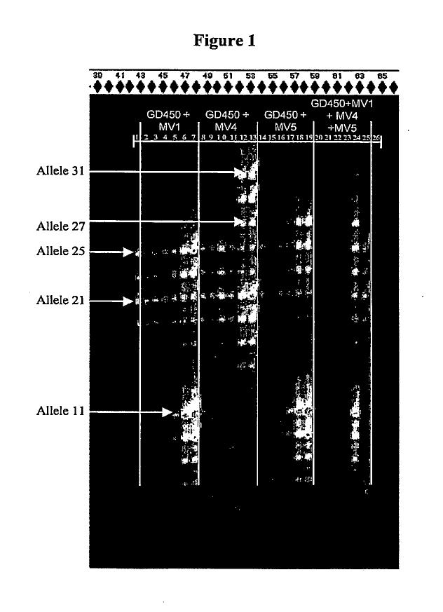

The results of PCR amplification of various mixtures of cat GD450 genomic DNA and microsatellite PCR product contaminants (see Table 1) are shown in FIG. 1. Short alleles (few repeats) give a stronger fluorescent signal if the template is a PCR product possibly due to increased efficiency of amplification.

All samples tested contained equivalent amounts of genomic DNA from cat GD450 and various amounts of contaminant PCR product generated from one or more cats MV1, MV4, MV5 (Table 1). PCR amplification of genomic cat GD450 DNA alone generated the expected bands for alleles 25 and 21 (FIG. 1, lane 1). In the presence of low concentrations of the contaminant (10⁻¹⁵-10⁻⁹) the GD450 profile was the only profile present. At higher concentrations (10⁻⁶, 10⁻³, undiluted) both the GD450 and the contaminant profile were present, making it difficult, or impossible, to determine the correct GD450 profile.

In mixtures containing MV1 or MV5 microsatellite PCR products (Lanes 2-7, and 14-19, respectively) the contaminant profile was evident when undiluted PCR (Lanes 7 and 19, respectively) product was added as well as dilutions of 10⁻³ and 10⁻⁶ (Lanes 5, 6, and 17, 18, respectively). When MV4 was the source of the contaminant it was detected in only undiluted and 10⁻³ samples (Lanes 12 and 13). When all three contaminating PCR products were mixed (total of 3 μl of PCR product added) the amount of contaminant appeared to inhibit the PCR amplification (Lane 25) but at reduced concentrations (Lane 24) the profile consisted of a combination of the three individual profiles. In lane 24, the alleles (11 and 25) present in more than one template (MV1+MV5 and MV1+GD450, respectively) give stronger signals than alleles 31, 27, 21 present in only one template due to the presence of multiple products.

These results clearly demonstrate that contaminating microsatellite PCR products are efficiently amplified during subsequent amplification for microsatellites from genomic DNA of cat GD450. In some of these mixtures subsequent PCR amplification resulted in a combination of the individual profiles such that the correct profile of test genomic DNA from cat GD450 is effectively masked. There is a clear relationship between the amount of contaminating microsatellite PCR product added to the genomic DNA and the amplification of the contaminant, with less contaminant resulting in reduced amplification of the contaminant in subsequent PCR amplifications. The amount of contaminant required to achieve this masking effect is extremely small. The undiluted contaminant had a concentration of approximately 50 ng/μl. At a dilution of 10⁻⁶ only 50 femtograms of PCR product was present. Since a typical PCR reaction would contain approximately 1 ng -100 ng of genomic DNA it is clear that only trace amounts of contaminating PCR product are required to mask the genuine GD450 profile.

### Example 2

### Demonstration That Contaminating Microsatellite PCR Products are Extracted With Genomic DNA and Efficiently Amplified During Amplification of Genomic DNA Microsatellite Loci

Materials and Methods

Template genomic DNA was isolated from muscle tissue of feral cat. Genomic DNA was isolated from cat GD450 that scores 25/21 at the locus FCA 69H (GenBank AF130500) carried by the cat chromosome B4 or from feral cat MV5 which gives 27/11 at the same locus. The contaminant for use in these experiments was produced by PCR amplification of selected microsatellite loci from DNA of cat MV5 and diluted with water to give a final concentration of 20 ng/μl.

PCR conditions and primer sequences used in this study are given in Menotti-Raymond, M.; David, V. A.; Lyons, L. A.; Schaffer, A. A.; Tomlin, J. F.; Hutton, M. K.; O'Brien, S. J. (1999). A Genetic Linkage Map of Microsatellites in the Domestic Cat (Felis catus). Genomics 57 (1), 9-23. Medline 99208656.

Extraction of Genomic DNA and Contaminant Removal

The genomic DNA of the cat GD450 was extracted from 4.5 mg of muscle tissue with the MasterPure™ DNA Purification Kit (Epicentre Technologies, Madison, Wis., US) using the modified protocol described in Experiment One. The Masterpure kit provided a more stringent DNA extraction method than the phenol or Chelex™ extraction methods recommended in forensic kits such as the AmpFISTR Profiler Plus™ Kit from Applied Biosystems and should result in less carry through of any contamination during the genomic DNA extraction procedure.

Seven different reaction tubes were setup:
1. Tissue (cat GD450) alone to obtain non contaminated genomic DNA profile
2. 5 ul (100 ng) of cat MV5 contaminant DNA alone
3. Tissue (cat GD450)+5 ul (100 ng) of MV5 contaminant
4. Tissue (cat GD450)+5 ul (100 ng) of MV5 contaminant digested with Hae III (Promega)
5. Tissue (cat GD450)+5 ul (100 ng) of MV5 contaminant digested with DNAse I (Sigma)
6. Tissue (cat GD450)+5 ul (100 ng) of MV5 contaminant treated with DNAZap (Ambion)
7. Tissue (cat GD450)+5 ul (100 ng) of MV5 contaminant washed with water.

In seven 1.5 ml microfuge tubes, cat GD450 tissue and/or cat MV5 microsatellite PCR product contaminant were mixed and left in contact for 30 min at room temperature. In tubes 1, 2, and 3 no further treatment was performed. In tube 4, 10× Promega Buffer B (3 μl), 0.5 μl Promega Hae III restriction enzyme (9 units) and 21.5 μl of water were added and incubated at 37° C. for 1 hour. In tube 5, 10× Promega Buffer C (3 μl), 0.5 μl DNAse I (˜50 units) and 21.5 μl water were added and incubated at 37° C. for 1 hour. In tube 6, DNAZap Solution 1 (10 μl) was added to the tube immediately followed by 10 μl of DNAZap Solution 2 (Ambion Pty Ltd). After approximately 10 seconds the tissue sample was thoroughly rinsed with deionised water prior to further use. In tube 7, the tissue sample was washed twice 1 ml of water, dried with tissue paper and transferred into a new tube. The tissue was then washed again with 2×1 ml of water.

In every tube, the integrity of the tissue samples was preserved following the treatments.

Following the above treatments 300 μl of Masterpure Tissue and Cell Lysis Buffer+50 μg Proteinase K were added (Epicentre Technologies). The samples were incubated overnight at 65° C. in a hybridization oven with rotation. After this treatment, no tissue was left in the tubes. RNA was removed by addition of 5 μg of RNAse A and incubation at 37° C. for 30 min. Tubes were cooled on ice and 150 μl of MPC Protein Precipitation Reagent were added (Epicentre Technologies). The precipitate was pelleted by centrifugation at 10,000 g for 10 minutes at 4° C. Supernatants were transferred into new tubes.

After addition of 500 μl of isopropanol and centrifugation for 10 minutes, the DNA pellet was washed with 70% EtOH and resuspended in 40 μl of water.

PCR Reactions

Samples from tubes 1-7 were used in individual PCR amplifications to detect microsatellite loci from cat GD450. Amplifications of individual microsatellite loci were performed in either 10 μl or 20 μl reactions containing 1 μl of template nucleic acid solution from each of tubes 1-7 according to the procedure of Menotti-Raymond et al (1999). The 10 μl reactions were approximated to contain 2.5 ng of contaminant (1/40×100 ng) whilst the 20 μl reactions contained approximately 10 ng of contaminant (4×2.5 ng).

A PCR amplification control was performed where 1 ul of contaminant (20 ng) was reamplified as above.

Results

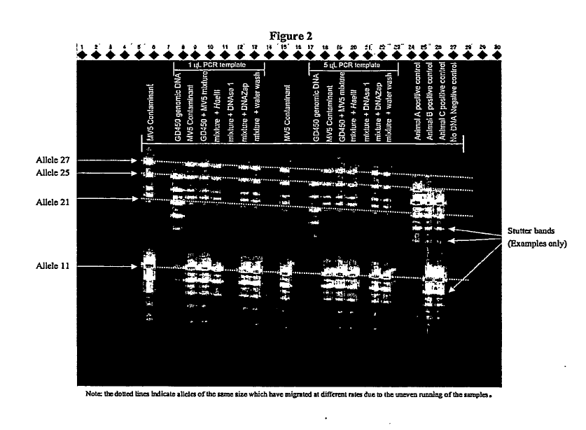

The results (FIG. 2) demonstrate the presence of the substantially correct profiles for MV5 contaminant and GD450 controls (Contaminant and Sample 1 lanes, respectively) as well as animal positive controls (animals A, B and C). Significant stutter peaks were present in the MV5 contaminant controls equivalent to alleles 25, 23 and 9. Additionally the MV5 contaminant control contained a band at a size equivalent to an allele at 22. The exact identity of this band is unknown but it is possibly the result of heteroduplex formation during PCR. In the GD450 positive control stutter peaks at allele equivalent 23, 19 and 17 are present. Smaller are bands in GD450 at allele equivalents 11 and 9 are the result of spillage from adjacent lanes.

In samples containing mixtures of cat GD450 genomic DNA and MV5 microsatellite PCR product contaminant (mixture samples) only bands representative of the MV5 contaminant were present. Since the contaminant amplified in these samples was added to cat GD450 tissue prior to genomic DNA extraction, the results show that contaminating microsatellite PCR products were efficiently extracted during the isolation of DNA from tissue of cat GD450. These PCR product contaminants could be amplified during subsequent PCR implication for cat GD450 microsatellite loci. The results further show that when 10 ng of contaminating microsatellite was added to 4.5 mg of tissue the contaminating microsatellite PCR products were able to entirely mask the genuine cat GD450 profile (eg: mixture samples).

With the exception of DNAse (mixture+DNAse) the treatments trialled to remove the contaminating microsatellite PCR products failed to have any significant effect on the level of contaminant amplified during subsequent amplification for cat GD450 microsatellite loci. In the sample treated with DNAse I, however, the microsatellite contaminant was efficiently removed since no detectable cat MV5 bands were present following PCR amplification for cat GD450 microsatellite loci (mixture+DNAse I, 1 μL and 5 μL samples). However, there was also an absence of GD450 bands suggesting that the treatment used also completely removed genomic DNA from the sample.

This result demonstrated that the removal of contaminating microsatellite PCR products from tissue is possible. However, it is not easily achieved by either physical or chemical methods that are frequently used to remove contaminating DNA. It also showed that the complete removal of contaminating microsatellite PCR products requires additions/modifications to both reagents and protocols in DNA extraction methods often used for DNA fingerprinting studies. DNAse I was effective at removing contaminating microsatellite PCR products but the method used in this study is not suitable for inclusion in a DNA extraction kit.

### Example 3

### Contamination of Human Nucleic Acid Samples

To confirm the contamination issue also affected other PCR DNA template and primer sets, particularly those involving human samples similar to typical forensic samples, experiments were conducted which used human buccal swabs.

Materials/Methods

In these experiments commercially available buccal swabs, typically used for parentage, pathogy and also forensic purposes were used to collect cheek cells from a human source. These buccal swabs were transported to a separate laboratory where they were impregnated with previously prepared mixture of PCR amplicons derived from DNA isolated from an unrelated human source. This mixture consisted of PCR amplicons derived from 6 separate microsatellite loci (FIBRA, D8S1179, D5S818, D7S820, D13S317, D19S253).

The human PCR amplicons were diluted with water to a concentration of 10 ng/μl and 1 μl of the undiluted as well as 10⁻³, 10⁻⁶, and 10⁻⁹ dilutions were prepared and separately added to individual buccal swabs. Where appropriate these swabs were used to collect cheek and other cells by wiping on the inside of the cheek as per standard operational procedures for collecting buccal samples prior to the addition of the contaminating PCR amplicons. DNA was then extracted from the material associated with the swabs using a rapid DNA extraction procedure known to work well with buccal swab samples for parentage analysis.

The purified DNA was then subjected to PCR amplification using primers specific for the above loci (FIBRA, D8S1179, D5S818, D7S820, D13S317, D19S253) using standard well established conditions for the amplification of these loci.

Results

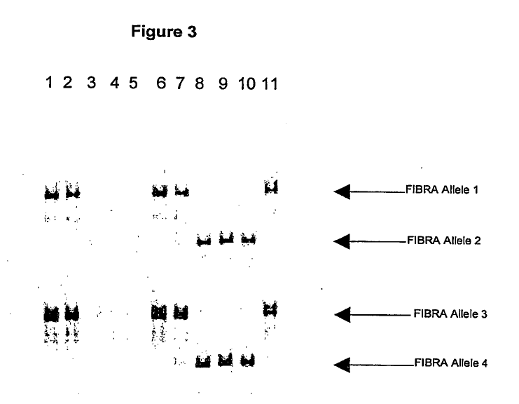

The results are depicted in FIG. 3. Samples (see Table hereunder) were subjected to DNA extraction and FIBRA locus specific amplification as indicated. Lanes 1-4 were samples with PCR amplicon dilutions only. The PCR amplicons contained only alleles 1 and 3 (Lane 11). Lanes 6-9 are amplification products from DNA extracts containing both cellular and contaminating PCR amplicons. The cellular DNA had a profile consisting of alleles 2 and 4 (Lane 10).

| | |
|---|---|
| Lane 1 | Swab containing undiluted PCR amplicons only. |
| Lane 2 | Swab containing 10⁻³ dilution of amplicons only. |
| Lane 3 | Swab containing 10⁻⁶ dilution of amplicons only. |
| Lane 4 | Swab containing 10⁻⁹ dilution of amplicons only. |
| Lane 5 | BLANK |
| Lane 6 | Swab containing cells and undiluted PCR amplicons. |
| Lane 7 | Swab containing cells and 10⁻³ dilution of amplicons. |
| Lane 8 | Swab containing cells and 10⁻⁶ dilution of amplicons. |
| Lane 9 | Swab containing cells and 10⁻⁹ dilution of amplicons. |
| Lane 10 | Swab containing cells only as positive control (alleles 2 |
| | and 4). |
| Lane 11 | Re-amplified PCR amplicons only (alleles 1 and 3). |

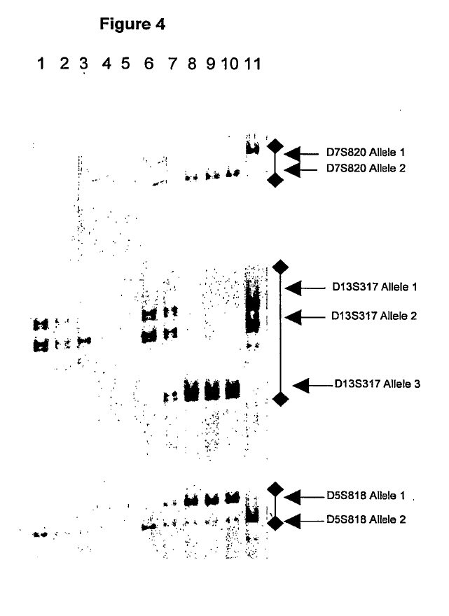

The results of the next procedure are depicted in the FIG. 4. Samples (see Table hereunder) were subjected to DNA extraction and a tri-plex amplification reaction with primers specific for the D5S818, D7S820, and D13S317 loci was performed as indicated. Lanes 1-4 were samples with PCR amplicon dilutions only. The PCR amplicons contained D7S820 allele 1, D13S317 alleles 1 and 2, D5S818 allele 2 (Lane 11). Lanes 6-9 contains amplification products from samples with both cellular DNA and contaminating PCR amplicons. The cellular DNA had a profile consisting of D7S820 allele 2, D13S317 allele 3, D5S818 allele 1 (Lane 10).

| | |
|---|---|
| Lane 1 | Swab containing cells and undiluted PCR amplicons. |
| Lane 2 | Swab containing cells and 10⁻³ dilution of amplicons. |
| Lane 3 | Swab containing cells and 10⁻⁶ dilution of amplicons. |
| Lane 4 | Swab containing cells and 10⁻⁹ dilution of amplicons. |
| Lane 5 | BLANK |
| Lane 6 | Swab containing cells and undiluted PCR amplicons. |
| Lane 7 | Swab containing cells and 10⁻³ dilution of amplicons. |
| Lane 8 | Swab containing cells and 10⁻⁶ dilution of amplicons. |
| Lane 9 | Swab containing cells and 10⁻⁹ dilution of amplicons. |
| Lane 10 | Swab containing cells only as positive control. |
| Lane 11 | Re-amplified PCR amplicons only. |

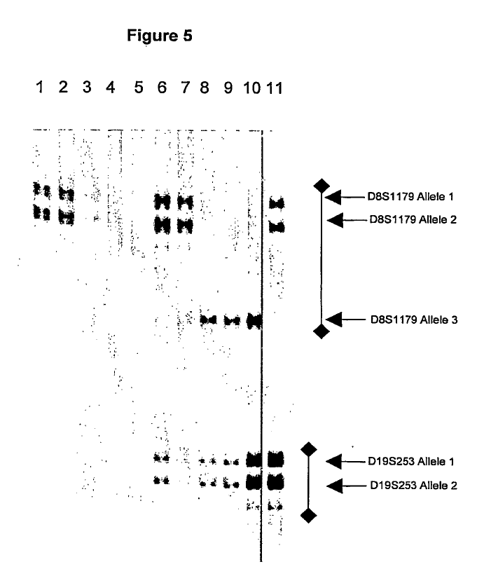

The results of the next procedure are depicted in the FIG. 5. Samples (see Table hereunder) were subjected to DNA extraction and a bi-plex amplification reaction with primers specific for the D8S1179 and D19S253 loci was performed as indicated. Lanes 1-4 were samples with PCR amplicon dilutions only. The PCR amplicons contained only D8SI 179 alleles 1 and 2, D19S253 alleles 1 and 2 (Lane 11). Lanes 6-9 contains amplification products from samples with both cellular DNA and contaminating PCR amplicons. The cellular DNA had a profile consisting of D8S1179 allele 3, D19S523 alleles 1 and 2 (Lane 10).

| | |
|---|---|
| Lane 1 | Swab containing cells and undiluted PCR amplicons. |
| Lane 2 | Swab containing cells and 10⁻³ dilution of amplicons. |
| Lane 3 | Swab containing cells and 10⁻⁶ dilution of amplicons. |
| Lane 4 | Swab containing cells and 10⁻⁹ dilution of amplicons. |
| Lane 5 | BLANK |
| Lane 6 | Swab containing cells and undiluted PCR amplicons. |
| Lane 7 | Swab containing cells and 10⁻³ dilution of amplicons. |
| Lane 8 | Swab containing cells and 10⁻⁶ dilution of amplicons. |
| Lane 9 | Swab containing cells and 10⁻⁹ dilution of amplicons. |
| Lane 10 | Swab containing cells only as positive control. |
| Lane 11 | Re-amplified PCR amplicons only. |

The results demonstrate that the profiles of the cheek and other cells present on the buccal swabs differed from the contaminant PCR amplicons for all loci examined except D19S253 (FIG. 3—Lanes 10 and 11; FIG. 4—Lanes 10 and 11; FIG. 5—Lanes 10 and 11). This enabled simple identification of genuine profiles derived from the cellular material and the profile from any added PCR amplicons.

In order for amplification of the added PCR amplicons to occur they must be extracted with similar efficiency to the cellular DNA during DNA extraction procedures. The results show that PCR amplicons, when added to a typical forensic sample such as a buccal swab, are extracted using the common DNA extraction protocol used here and are efficiently amplified in subsequent PCR reactions for the appropriate loci. In all experiments, regardless of the locus amplified, the contaminating amplicons were able to completely mask the genuine profile of the cells in the buccal swab (FIG. 3—Lanes 6 and 7; FIG. 4—Lanes 6 and 7; FIG. 5—Lanes 6 and 7). This effect occurred with undiluted (10 ng/μl) and 10⁻³ (10 pg/μl) samples (FIG. 3—Lanes 6 and 7; FIG. 4—Lanes 6 and 7; FIG. 5—Lanes 6 and 7).

Very little or no visible contamination with added PCR amplicons was seen with 10⁻⁶ (10 fg/μl) and 10⁻⁹ (10 ag/μl) dilutions (FIG. 3—Lanes 8 and 9; FIG. 4—Lanes 8 and 9; FIG. 5—Lanes 8 and 9) and even when no cellular DNA was present (FIG. 3—Lanes 1 to 4; FIG. 4—Lanes 1 to 4; FIG. 5—Lanes 1 to 4). This indicates that the failure to amplify any contaminating PCR amplicons present in the sample is due to the extremely low level (very few amplicon molecules) of the original sample using the PCR conditions used here. The use of a more sensitive amplification and/or detection procedure may detect the low levels of contaminating PCR amplicons present in even these samples.

These results clearly demonstrate the potential for contaminating PCR amplicons to mask or confuse the DNA profile derived from a typical source of forensically important samples.

### Example 4

### Cleanup of Nucleic Acid Samples Using Micron Centricon Ultracentrifugation

To demonstrate the principle of ultrafiltration as an example of a physical means of removing contamination PCR amplicons from genomic DNA (or sample) Amicon YM-100, Microcon-100 microconcentrators (nominal molecular weight cut-off of 100 kDa, corresponding to 300 nt of single-stranded DNA or 125 bp of double-stranded DNA: Millipore Application note AN023EN00), were used to separate PCR amplicons from cellular genomic DNA.

Many small PCR amplicons such as oligonucleotides used for profiling with single nucleotide polymorphisms will pass directly through these microconcentrators. Other PCR amplicons such as those from typical forensic analyses including the commercially available DNA profiling kits (as well as many other microsatellite and mitochondrial DNA profiling analyses) are all larger than 125 bp. Consequently, this method will not separate genomic DNA templates from double stranded these larger amplified DNA products.

However, as the nominal molecular weight cut-off for single stranded DNA is approximately twice the double stranded DNA cut-off (YM-100; 300 and 125, respectively) denaturation of the template will allow many, and perhaps all (depending on the size of PCR amplicons) to pass through the microconcentrator, allowing separation of the contaminating PCR amplicons and high yield recovery of the important sample genomic DNA.

Methods

DNA was isolated from 2 cattle blood samples and subjected to PCR amplification using BM2113 microsatellite primers with the sequence;

5BM2113 forward -
(5′) GCT GCC TTC TAC CAA ATA CCC (3′)
BM2113 reverse -
(5′) CTT CCT GAG AGA AGC AAC ACC (3′).

The amplification reaction consisted of the following (per reaction),

| | |
|---|---|
| BM2113 forward primer: | 1 μl |
| BM2113 reverse primer: | 1 μl |
| 10× DNA polymerase buffer (Promega): | 2.5 μl |
| dNTPs (200□M each): | 4 μl |
| Magnesium chloride (25 mM): | 2.5 μl |
| Taq polymerase (Applied Biosystems): | 1 μl |
| distilled water: | 11 μl |
| Template DNA (approximately 100 ng): | 2 μl |
| TOTAL | 25 μl |

The reaction mix was then subjected to the following thermal cycle in an Applied Biosystems GeneAmp PCR System 9700 thermal cycler to amplify the template DNA present.

Denaturation: 95° C./15 minutes

Annealing and extension (31 cycles):

94° C./45 seconds (100% ramp rate)

61° C./45 seconds (50% ramp rate)

72° C./60 seconds (80% ramp rate)

Final extension: 72° C./60 minutes

Final Step: 25° C./2 hours

Hold: 4° C. until required.

The resultant amplification products were then separated by high resolution agarose gel electrophoresis (Fisher Biotec Ultra High Resolution Agarose) in 6% gels until the required separation was achieved.

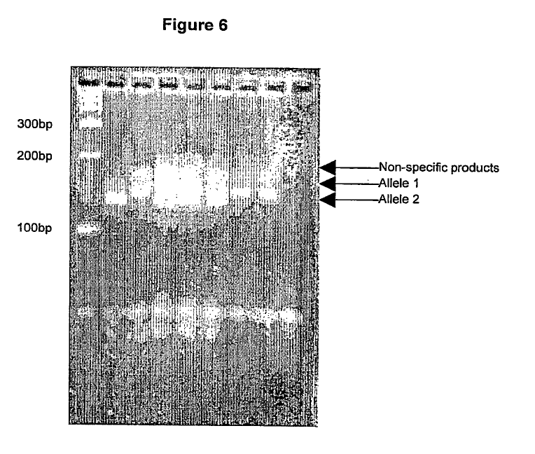

The cattle DNA samples used were amplified using the BM2113 primers to determine the alleles present in each animal (FIG. 6—Lanes 2 and 3). This showed the animals had easily differentiated loci. The PCR product (1 μl of 1/10,000 dilution of the PCR reaction, approximately 0.1 pg) from animal 1 (FIG. 6—Lane 2) were then added to approximately 10 ng of template genomic DNA from animal 2. The mixture of genomic DNA and PCR amplicons was then subjected to amplification as previously using BM22113 primers. Analysis of the resultant products (FIG. 6—Lane 4) demonstrated the presence of alleles from both animal 1 (allele 1) and animal 2 (allele 2).

To separate contaminating PCR amplicons and cellular genomic DNA by ultrafiltration, a Microcon-100 sample reservoir was placed into a microcentrifuge tube and the reservoir filled with 400 μl TE(10 mM Tris-HCl pH8.0, 0.1 mM EDTA) and up to 50 μl of untreated or treated amplicon/genomic DNA mixture. The treatments consisted of one of either, heat denaturation at 95° C. for 1-15 minutes in deionised water or formamide solution (10 mM NaOH, 95% deionised formamide), or alkaline denaturation with sodium hydroxide (0.2M) for 5 minutes followed by neutralisation.

After centrifugation at 500 g for 15 min in an Eppendorf microcentrifuge (Model 5415C) at room temperature, another 400 μl of TE was added to the sample reservoir and centrifugation continued for approximately 15 minutes or until the volume in the retentate cup was reduced to about 20 μl. To recover retained DNA larger than the nominal molecular weight cut-off, the reservoir was removed and inverted into a new microcentrifuge tube then centrifuged at 500 g for 2 min. The resulting DNA (approximately 20 μl) was used in subsequent PCR amplification studies with BM2113 to identify the source (contaminating amplicon or cellular DNA) of the DNA present in the concentrate solution.

Results

Without treatment to render the DNA single stranded, the PCR amplicons and cellular genomic DNA were efficiently retained in the concentrate (FIG. 6—Lanes 5 and 6). This is expected as the as the microconcetrators are designed to retain genomic DNA and the added PCR amplicons are approximately 140 bp in size whilst the cut-off of the microconcentrator membrane is approximately 125 bp.

However, treatment of the DNA mixture prior to PCR by heating at 95° C. for 10 minutes to render the DNA single stranded, followed by immediate application to the microconcentrator and centrifugation as above, resulted in almost complete removal of the added amplicon DNA (FIG. 6—Lanes 7 and 8). In FIG. 6 only the alleles representative of cellular genomic DNA are present (Lanes 7 and 8).

This clearly demonstrates that contaminating PCR amplicons can be removed by ultrafiltration using a microconcentrator with an appropriate nominal molecular weight cut-off. For amplicons with sizes that are not suited to the Amicon YM-100 Microcon-100 microconcentrators other microconcentrators with a suitable cut-off can be used.

### Example 5

### Cleanup of Nucleic Acid Samples Using Sodium Bisulfite

To demonstrate the principle of chemical modification of the DNA as an example of a chemical means of removing contamination PCR amplicons from genomic DNA (or sample) DNA was treated with sodium bisulfite. This reagent is commonly used to convert cytosine, but not 5-methylcytosine, in DNA into uracil. It is particularly useful for DNA methylation studies (Granau et al, Nucleic Acids Research (2001) Vol. 29, No. 13; Raizis et al, Analytical Biochemistry (1995) Vol. 226, 161-166.) This feature also makes it a useful reagent for the specific removal of PCR amplicons from genomic DNA as the PCR amplicons contain only cytosine (there is no methylation of cytosines during PCR) whilst the human cellular genomic DNA (and DNA from many other organisms) will contain a proportion of methylated cytosines which are susceptible to conversion to uracil.

Because of the specific interaction between cytosine and sodium bisulfite the conversion of cytosine to uracil under the conditions used proceeds very rapidly with single stranded DNA, but only slowly when the DNA is double stranded. Since PCR amplicons are relatively small compared to genomic DNA they are more readily denatured into single stranded form. This will facilitate the cytosine to uracil conversion in any PCR amplicons present whilst rendering the genomic DNA considerably more resistant to such conversion.

As demonstrated in laboratory contamination control of PCR amplicon contamination using uracil glycosylase, this enzyme is able to remove the uracil from DNA rendering the DNA incapable of amplification in subsequent PCR reactions.

By treating the converted DNA from the sodium bisulfite reaction with uracil glycosylase the contaminating PCR amplicons in a sample should similarly become resistant to subsequent amplification by PCR.

Methods

The methods and reagents used for the bisulfite treatment of DNA were essentially as described by Raizis et al (Analytical Biochemistry (1995) Vol. 226, 161-166). A 5M sodium bisulfite solution was prepared by adding 1.9 g of sodium metabisulfite to 2.5 ml of distilled water and vortexing for 1 minute. To this solution 0.7 ml of 0.2M sodium hydroxide and 0.5 ml of hydroquinone were added. The solution was vortexed until completely dissolved. The pH was adjusted to 5.0 with sodium hydroxide and the final volume was made up to 4 ml with distilled water.

To obtain single-stranded DNA, the DNA (approx 0.5 μg) was incubated in 0.3 M NaOH at 37° C. for 20 min. These conditions were chosen as initial studies demonstrated that PCR amplicons appeared to be preferentially denatured using these conditions. The reaction volume was adjusted to 30 μl with sterile water and 3 μl of 2 M sodium hydroxide was added. The DNA was incubated at 45° C. for 15 minutes and following this incubation freshly prepared bisulfite solution was added (200 μl/μg DNA) directly to the denatured DNA. The mixture was incubated at 50° C. for 4 hours.

The DNA was neutralised and precipitated by the addition of 0.5 volumes of 3M sodium acetate (pH7.0), 2.3 volumes of distilled water, and an equal volume of isopropanol. The mixture was incubated on ice for at least 30 minutes. The DNA was then pelleted by centrifugation in a microcentrifuge for 15 minutes.

The supernatant was discarded and the pellet was resuspended in 300 μl distilled water, 30 μl 3M sodium acetate (pH7.0) and reprecipitated with 2 volumes of ethanol. The DNA was then pelleted by centrifugation in a microcentrifuge for 15 minutes. The pellet was redissolved in 200 μl of sodium hydroxide (0.2M) and incubated at room temperature for 15 minutes.

The DNA was then precipitated by the addition of 0.5 volumes of 7.5M ammonium acetate and 2 volumes of ethanol. Following microcentrifugation for 30 minutes the supernatant was discarded and the DNA pellet was redissolved in 50 μl of distilled water. The DNA was then analysed by agarose gel electrophoresis or subjected to PCR amplification.

To demonstrate this method DNA was isolated from 2 cattle blood samples and subjected to PCR amplification using SPS 115 microsatellite primers. These have the sequence;

SPS 115 forward -
(5′) AAA GTG ACA CAA CAG CTT CTC CAG (3′)
SPS 115 reverse -
(5′) AAC GAG TGT CCT AGT TTG GCT TGT (3′).

The amplification reaction consisted of the following (per reaction),

| | |
|---|---|
| SPS 115 forward primer: | 1 μl |
| SPS 115 reverse primer: | 1 μl |
| 10× DNA polymerase buffer (Promega): | 2.5 μl |
| dNTPs (200□M each): | 4 μl |
| Magnesium chloride (25 mM): | 2.5 μl |
| Taq polymerase (Applied Biosystems): | 1 μl |
| distilled water: | 11 μl |
| Template DNA (approximately 100 ng) | 2 μl |
| TOTAL | 25 μl |

The reaction mix was then subjected to the following thermal cycle in an Applied Biosystems GeneAmp PCR System 9700 thermal cycler to amplify the template DNA present.

Where required Uracil-DNA-glycosylase (1 μl—Fisher Biotec) was added to the PCR reaction mix prior to the initial denaturation step and incubated at 37° C. for 15-60 minutes to digest any uracil containing DNA.

Denaturation: 95° C./15 minutes

Annealing and extension (31 cycles):

94° C./45 seconds (100% ramp rate)

61° C./45 seconds (50% ramp rate)

72° C./60 seconds (80% ramp rate)

Final extension: 72° C./60 minutes

Final Step: 25° C./2 hours

Hold: 4° C. until required.

The resultant amplification products were then separated by high resolution agarose gel electrophoresis (Fisher Biotec Ultra High Resolution Agarose) in 6% gels until the required separation was achieved.

Results

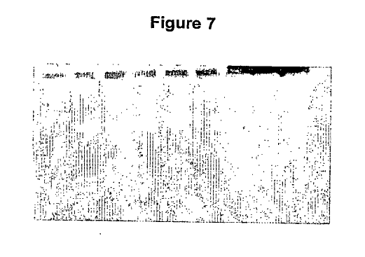

DNA isolated from animal 1 and animal 2, respectively was subjected to PCR amplification with SPS 115 primers. Animal 1 (FIG. 7—Lane 2) had only allele 2 whilst animal 2 had allele 1 and allele 2 (FIG. 7—Lane 3). These were readily differentiated using 6% high resolution agarose gel electrophoresis allowing discrimination of the source of the alleles (animal 1 or animal 2).

The PCR product (1 μl of 1/10,000 dilution of the PCR reaction, approximately 0.1 pg) from animal 2 (FIG. 7—Lane 3) were then added to approximately 100 ng of template genomic DNA from animal 1. The mixture of genomic DNA and PCR amplicons was then subjected to amplification as previously using SPS 115 primers. Analysis of the resultant products (FIG. 7—Lane 4) demonstrated the presence of alleles from both animal 1 (allele 1) and animal 2 (allele 2).

To separate contaminating PCR amplicons and cellular genomic DNA, the amplicon/genomic DNA mixture above treated with bisulfite and uracil-DNA-glycosylase according to the methods above. In these studies the PCR amplicons were diluted 1/1000 and 1 μl was added to approximately 1 μg of template genomic DNA. This was to compensate for potential losses of DNA during the processing with bisulfite.

The DNA mix was treated with bisulfite with (FIG. 7—Lanes 7 and 8) or without (FIG. 7—Lanes 5 and 6) initial template DNA denaturation. In samples that were not denatured both alleles 1 and 2 were present following amplification/digestion (FIG. 7—Lanes 5 and 6). There appeared to be little loss of allele 1 associated exclusively with animal 2. As allele 1 was present in the sample only through the addition of contaminating PCR amplicons derived from animal 2 this demonstrates that there had been little detectable loss of the contaminating PCR amplicons from the mixture.

In contrast where the sample template DNA had been denatured prior to sodium bisulfite treatment, following PCR amplification/digestion, there was significant loss of allele 1 (FIG. 7—Lanes 7 and 8) although allele 2 was still present at detectable levels. This suggests that the treatment had resulted in the preferential removal of contaminating PCR amplicons from the mixed sample.

The results show that it is possible to use sodium bisulfite in combination with uracil-DNA-glycosylase to effectively remove contaminating PCR amplicons.

The present invention includes modifications and adaptations apparent to those skilled in the art.

Throughout the specification, unless the context requires otherwise, the word “comprise” or variations such as “comprises” or “comprising”, will be understood to imply the inclusion of a stated integer or group of integers but not the exclusion of any other integer or group of integers.

1. A method of analysing a nucleic acid sample obtained from a site comprising the step of pretreating the sample to remove or inactivate contaminating nucleic acids originating from the site.

2. A method according to claim 1 wherein the contaminating nucleic acid is deoxyribonucleic acid (DNA), ribonucleic acid (RNA), locked nucleic acid (LNA) or protein nucleic acid (PNA).

3. A method according to claim 1 wherein the contaminating nucleic acid is particularly well adapted for amplification via PCR or some other amplification process.

4. A method according to claim 3 wherein the contaminating nucleic acid is an amplicon derived from a PCR or another DNA amplification process.

5. A method according to claim 1 wherein the contaminating nucleic acid is degradation resistant.

6. A method according to claim 1 wherein the contaminating nucleic acid is synthetic.

7. A method according to claim 1 wherein the pre-treatment comprises treating the sample to preferentially remove or inactivate nucleic acids that are free or substantially free from other cell components.

8. A method according to claim 7 wherein the pre-treatment is one or more treatments selected from the group comprising: (i) enzymic treatments; (ii) physical treatments; and (iii) chemical treatments.

9. A method according to claim 8 wherein the enzymic treatments comprise contacting the sample with DNAses, RNAses, exonucleases and/or 25 endonucleases.

10. A method according to claim 8 wherein the physical treatments comprise centrifugation, washing, filtration and/or chromatography such as gel filtration chromatography.

11. A method according to claim 8 wherein the chemical treatments comprise the use of sodium hydroxide, sodium hypochlorite, sodium metabisulphite or ammonium metabisulphite, detergents and/or proprietary products designed to remove nucleic acids form surfaces.

12. A method according to claim 1 wherein the method of analysing the nucleic acid sample is PCR, mitochondrial DNA sequencing, single nucleotide polymorphism (SNP) analysis and low copy number PCR.

13. A method according to claim 1 wherein the pre-treatment comprises removing cell bound contaminating nucleic acids from the sample.

14. A method according to claim 13 wherein the cell bound contaminating nucleic acid is particularly well adapted for amplification via PCR or some other amplification process.

15. A method according to claim 13 wherein the contaminating nucleic acid is of bacterial origin.

16. A method according to claim 15 wherein the contaminating nucleic acid is bacteria engineered to contain at least one multicopy plasmid comprising at least one amplicon.

17. A method according to claim 13 wherein the cell bound contaminating nucleic acid is removed by exposing the nucleic acid in the cells and then removing the nucleic acid.

18. A method according to claim 17 wherein the nucleic acid is exposed by lysing the cells.

19. A method according to claim 17 wherein the nucleic acid is removed using the pre-treatment steps of claim 7.

20. A nucleic acid analysis kit comprising a means to remove a nucleic acid contaminant from a sample to be subjected to analysis.

21. A kit according to claim 20 wherein said means comprises a labelled probe adapted to bind to the contaminant and thus aid in its removal.

22. A kit according to claim 20 wherein said means comprises an enzyme or chemical that can be added to the sample and inactivate of remove the contaminant preferentially or selectively relative to a target nucleic acid.

23-42. (canceled)
